(19) United States
(12) Patent Application Publication
(10) Pub. No.: US 2024/0252792 A1
(43) Pub. Date: Aug. 1, 2024

(54) IMPLANTABLE MEDICAL DEVICES HAVING CANISTERS ENCLOSED IN ANCHORS WITH CAVITIES, AND ASSOCIATED SYSTEMS AND METHODS

(71) Applicant: Shifamed Holdings, LLC, Campbell, CA (US)

(72) Inventors: Scott Robertson, San Jose, CA (US); Anthony Pantages, San Jose, CA (US); Brian Fahey, Menlo Park, CA (US)

(21) Appl. No.: 18/560,611

(22) PCT Filed: May 20, 2022
(86) PCT No.: PCT/US2022/030380

§ 371 Date: Nov. 13, 2023

Related U.S. Application Data
(60) Provisional application No. 63/191,618, filed on May 21, 2021.

(51) Int. Cl. A61M 27/00

(52) U.S. Cl. CPC A61M 27/002; A61M 2205/0266; A61M 2205/3303; A61M 2205/3327; A61M 2205/35; A61M 2205/8206

(57) ABSTRACT

The present technology includes implantable medical devices having a first disc-shaped anchoring portion and a second disc-shaped anchoring portion connected to the first disc-shaped anchoring portion by an intermediate neck region. The first and second disc-shaped anchoring portions can be formed by one or more filaments woven or formed in a mesh-like pattern. The filaments can be configured such that the first and second disc-shaped anchoring portions have three-dimensional bodies having a partially enclosed cavity or chamber. The device can further include one or more canisters positioned within and retained by the partially enclosed chamber. The canisters can house one or more components of the device, such as an energy storage component, a sensor, or other electronics.

### CROSS REFERENCE TO RELATED APPLICATION(S)

This application claims the benefit of U.S. Provisional Patent Application No. 63/191,618, filed May 21, 2021, and incorporated by reference herein in its entirety.

### TECHNICAL FIELD

The present technology is generally directed to implantable medical systems and devices and, in particular, to implantable medical devices having canisters enclosed in cavities of anchors formed from a plurality of filaments.

### BACKGROUND

Implantable medical devices often include components that need to be shielded from direct contact with biological tissue or fluid. For example, various medical devices may include electronics (e.g., sensor electronics, device control electronics, communication electronics, etc.) and/or energy storage devices (e.g., batteries, capacitors, supercapacitors, etc.) for powering one or more active components of the device (e.g., sensor(s), actuator(s), etc.). To protect the electronics from biological tissue, the electronics can be placed in a hermetically sealed canister. However, delivering such canisters through a catheter during deployment of the device can be challenging. Moreover, once implanted, the canisters, if not secured to the device appropriately, could detach from the device and pose a threat to the patient's health.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A is an isometric view of an implantable medical device configured in accordance with select embodiments of the present technology.

FIG. 1B is a side view of the implantable medical device of FIG. 1A.

FIG. 2 is a schematic illustration of the device of FIGS. 1A and 1B implanted across a septal wall of a patient's heart in accordance with select embodiments of the present technology.

### DETAILED DESCRIPTION

The present technology is generally directed to implantable medical devices and associated systems and methods. In some embodiments, the implantable medical devices include a first disc-shaped anchoring portion and a second disc-shaped anchoring portion connected to the first disc-shaped anchoring portion by an intermediate neck region. The first and second disc-shaped anchoring portions can be formed by one or more filaments. For example, the one or more filaments can include one or more braided wires woven or otherwise formed in a mesh-like pattern. Alternatively, the filaments may include multiple elongate filaments cut from a single tube or sheet that are subsequently formed into disc-shapes. The filaments can be braided or otherwise formed such that the first and second disc-shaped anchoring portions have three-dimensional bodies having an at least partially enclosed cavity or chamber. The device can further include one or more canisters positioned within and retained by the partially enclosed chamber. The canisters can house one or more components of the device, such as an energy storage component, a sensor, or other electronics. Without being bound by theory, retaining the canisters within a chamber formed by the first anchoring portion and/or the second anchoring portion is expected to reduce the complexity of deploying the device from a catheter and/or reduce the risk of the canisters detaching from the device after deployment within the patient.

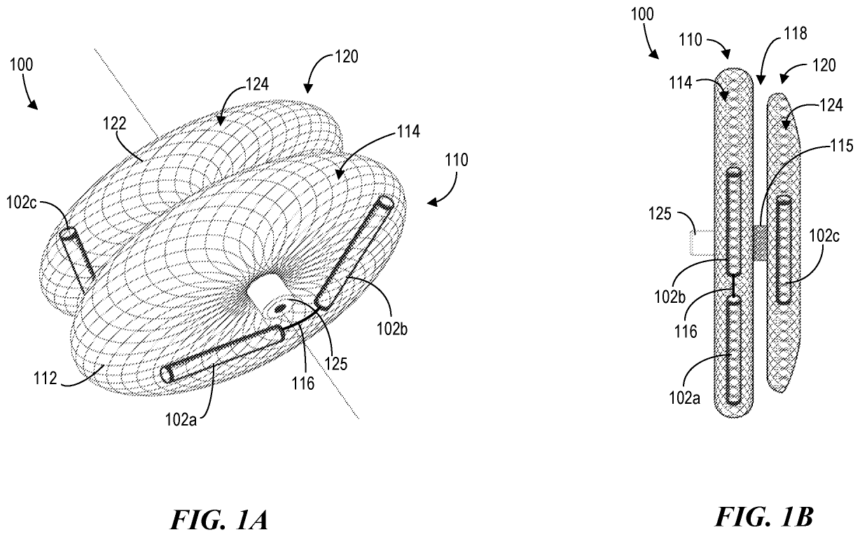

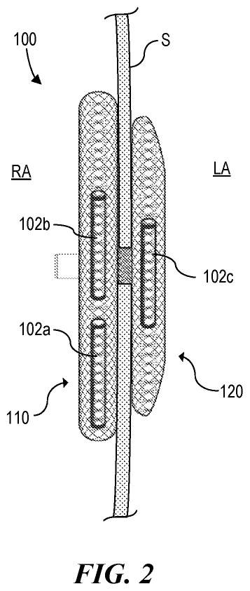

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1A-2.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the terms “chamber,” “cavity,” or the like refer to a volume of space defined by a structure that can retain another element. These terms do not require the volume to be fully enclosed, unless specifically noted otherwise.

FIG. 1A is an isometric view of an implantable medical device 100 (“the device 100”) configured in accordance with select embodiments of the present technology, and FIG. 1B is a side view of the device 100. Referring to FIGS. 1A and 1B collectively, the device 100 includes a first umbrella or anchoring portion 110 and a second umbrella or anchoring portion 120. The first anchoring portion 110 and the second anchoring portion 120 are connected by an intermediate portion 115 (also referred to as a “neck” portion) extending between the first anchoring portion 110 and the second anchoring portion 120. The first anchoring portion 110 and the second anchoring portion 120 can be generally disc-shaped, toroid-shaped, or the like, such that they define a three-dimensional body having a partially enclosed first internal chamber or cavity 114 and a partially enclosed second internal chamber or cavity 124, respectively. Although shown to be larger than second anchoring portion 120, in some embodiments the first anchoring portion 110 may be the same size as or smaller than the second anchoring portion 120.

The first anchoring portion 110 and the second anchoring portion 120 may be at least partially separated by a gap 118 whose geometry is defined by the size and shape of the neck portion 115, first anchoring portion 110, and second anchoring portion 120. As described in greater detail below; the gap 118 can be configured to receive patient tissue when the device 100 is implanted and deployed within a patient. For example, once the device 100 is deployed at a target location within a patient, the device 100 is anchored to patient tissue at the target location by the first anchoring portion 110 and the second anchoring portion 120 retaining tissue therebetween in the gap 118. The height of the gap 118 is defined by the difference in an outer diameter of the first anchoring portion 110 or the second anchoring portion 120 and an outer diameter of the intermediate portion 115. The height of the gap 118 can therefore be varied (e.g., during manufacturing, during heat-setting, etc.) by changing an outer diameter of at least one of the anchoring portions 110, 120 and/or the intermediate portion 115. Likewise, the width of the gap 118 can be varied (e.g., during manufacturing) by changing the length of the intermediate portion 115. Accordingly, the device 100 can be designed to accommodate different volumes of tissue when deployed in a patient depending on the desired application of the device 100, and is not limited to the configuration depicted in FIGS. 1A and 1B. For example, in some embodiments, the first and/or second anchoring portions 110, 120 are not disc shaped (e.g., the first and/or second anchoring portions 110, 120 can be square, pyramid, or otherwise shaped). In some embodiments, the first and/or second anchoring portions 110, 120 are not symmetric about a central axis. In some embodiments, the first and/or second anchoring portions 110, 120 can be configured such that they do not actively serve functionally as anchoring features. In such embodiments the device 100 can be fixated and/or stabilized via an alternate technique, or not be actively fixated/stabilized at all. In such embodiments, the three-dimensional elements referred to herein as the first and second anchoring portions 110, 120 can be configured to hold canisters as described below but without directly interfacing with patient tissue. Further, in some embodiments the gap 118 can be defined by alternative landmarks associated by aspects of the device.

The first anchoring portion 110 can be formed by one or more first filaments 112 (e.g., wires, braided wires, threads, etc.) braided/woven in a mesh-like pattern, and the second anchoring portion 120 can be formed by one or more second filaments 122 (e.g., wires, braided wires, threads, etc.) also braided/woven in a mesh-like pattern. The filaments 112, 122 can each include one, two, three, four, five, six, or more individual filaments woven together. In some embodiments, the filaments 112, 122 are formed by a single contiguous filament woven back and forth upon itself to form both the first anchoring portion 110 and the second anchoring portion 120. In some embodiments, one or more of the filaments 112, 122 that form the first anchoring portion 110 and/or the second anchoring portion 120 can also form the intermediate portion 115 connecting the first anchoring portion 110 and the second anchoring portion 120. End segments of the filaments 112, 122 can be connected/secured by a cap or holder 125. Although the device 100 is shown as having one cap 125, in other embodiments the device 100 may have more than cap 125 (e.g., a first cap positioned adjacent the first anchoring portion 110 and a second cap positioned adjacent the second anchoring portion 120). In alternative embodiments, the end segments of the filaments can be secured without the use of a cap or holder, for example they can be affixed/adhered to sections of the braided structure using soldering, crimps, adhesives, sutures, or other methods known to those skilled in the art. In some embodiments the first anchoring portion 110 and second anchoring portion 120 can be formed from a solid tube or sheet whereby individual first filaments 112 and second filaments 122 are manufactured (e.g., via laser-cutting, chemical etching, waterjet cutting, machining, etc.) to produce a continuous unibody. The first filaments 112 and the second filaments 122 may be further manipulated (e.g., by cold forming, shape-setting, etc.) to produce the first anchor portion 110 and the second anchor portion 120 that includes the first cavity 114 and the second cavity 124.

The filaments 112, 122 can be composed of any suitable material, such as Nitinol or another biocompatible material. In some embodiments, the filaments 112, 122 are composed of a shape-memory material having superelastic properties at body temperature to assist in the de-catheterization and deployment of the device (e.g., such that when released from a delivery catheter/delivery sheath, the device 100 automatically assumes the configuration shown in FIGS. 1A and 1B). In some embodiments, the filaments 112, 122 are composed of a material (or include a coating) that promotes endothelialization or other tissue overgrowth of the filaments 112, 122 once the device 100 is implanted to further assist in securing the device 100 in a desired location.

The device 100 further includes a plurality of canisters 102a-c (also referred to herein as “cans 102”). The cans 102 can include a fluidically- and/or hermitically-sealed housing that defines an internal compartment (not shown) for storing one or more components of the device 100. For example, the cans 102 can house one or more energy storage components (e.g., a battery, a capacitor, a supercapacitor, etc.), electronics (e.g., control circuitry, communication circuitry, sensor circuitry, etc.), one or more sensors (e.g., blood pressure sensors, heart rate sensors, blood oxygenation sensors, etc.), or the like. Each of the cans 102 can store the same or different components, and may have the same or different dimensions. In some embodiments, two or more of the cans 102 can optionally be connected via an electrical lead 116 or via another mechanical mechanism, for example with electronic leads to communicate electrical signals, energy, or other relevant transmissions from components of one canister to components of another canister. In some embodiments, the cans 102 can be formed of an anti-thrombogenic and/or biocompatible material, and/or can include an antithrombogenic and/or biocompatible coating.

Of note, the cans 102 are positioned within, and thus retained by, the first chamber 114 defined by the first anchoring portion 110 and/or within the second chamber 124 defined by the second anchoring portion 120. In the illustrated embodiment, for example, a first can 102a and a second can 102b are positioned within the first chamber 114 defined by the first anchoring portion 110, and a third can 102c is positioned within the second chamber 124 defined by the second anchoring portion 120. Although shown as having two cans 102 in the first chamber 114 and one can 102 in the second chamber 124, the device 100 can have a different number of cans 102 in the first chamber 114 and the second chamber 124, such as between zero to ten or more cans 102 in each of the first chamber 114 and the second chamber 124.

In the illustrated embodiment, the chambers 114, 124 are enclosed such that the canisters 102 cannot escape from the chambers 114, 124, but the chambers 114, 124 are not fluidly isolated from biological fluid (e.g., blood or other fluid can flow through the mesh-like pattern of the filaments 112, 122 and into the chambers 114, 124). Accordingly, in some embodiments the first anchoring portion 110 and/or the second anchoring portion 120 optionally include a membrane or fabric (not shown) coupled to the filaments 112, 122. The membrane or fabric can partially or fully fluidly isolate the chambers 114, 124 from an environment external to the device 100 (e.g., to prevent blood or other fluid from entering the chambers 114, 124). In such embodiments, the cans 102 can therefore also be fluidly isolated from an environment external to the device 100. The membrane or fabric can promote a physiologic response when implanted (e.g., promote tissue over-growth, inhibit tissue over-growth, or control tissue over-growth to a prescribed thickness or cell type).

By positioning the cans 102 in the chambers 114, 124 formed by the anchoring portions 110, 120, the cans 102 are retained by the device 100, and thus are not at risk of inadvertently detaching from the device 100 and circulating through the patient's vasculature or other body systems (e.g., which may cause a blockage or other health risk). In some embodiments, the cans 102 are optionally secured to the first anchoring portion 110 and/or the second anchoring portion 120 even though the cans 102 are positioned within the chambers 114, 124. The cans 102 can be secured to the corresponding anchoring portion via a tether, suture, snap-fit, or other suitable mechanism. This can help further ensure the orientation of the cans 102 relative to other aspects of the device 100 when the device 100 is implanted. In other embodiments, the cans 102 are positioned within the chambers 114, 124 but are not otherwise secured or tethered thereto.

Positioning the cans 102 in the chambers 114, 124 is also expected to simplify deploying the device 100 from a catheter, sheath, or other delivery system. For example, because the cans 102 reside within the chambers 114, 124, the device 100 can be deployed in any orientation with little to no risk of the cans blocking or otherwise interfering with various other aspects of the device 100 or patient tissue once deployed.

The device 100 can be configured for use in a variety of different medical applications. In one such application, for example, the first anchoring portion 110 can be positioned in a first body region and the second anchoring portion 120 can be positioned in a second body region separate from the first body region. For example, FIG. 2 is a schematic illustration of the device 100 implanted in the atrial septum such that the septal wall S resides within the gap 118 (FIG. 1B), the first anchoring portion 110 is positioned in a right atrium RA of the patient's heart (or, alternatively, the left atrium), the second anchoring portion 120 is positioned in the left atrium LA of the patient's heart (or, alternatively, the right atrium), and the intermediate portion 115 extends through a puncture or hole in the septal wall S. In such embodiments, the cans 102 in the first anchoring portion 110 will be positioned in the right atrium RA while the cans 102 in the second anchoring portion 120 will be positioned in the left atrium LA. The general H-shape of the device 100 in a side view (FIGS. 1B and 2) secures it to the septal wall S to prevent or at least reduce unwanted migration of the device 100.

In some embodiments, the device 100 is an implantable sensing device configured to measure one or more physiologic parameters of a patient. For example, the device 100 can be implanted across a patient's atrial septum as described above, and can be configured to measure right atrial pressure and/or left atrial pressure. In such embodiments, the cans 102 carry sensor(s), sensor power source(s), sensor electronics, or the like. In addition to or in lieu of being a sensing device, the device 100 may also function as an occlusion device, an anastomosis device, a shunting device, or the like. In embodiments in which the device 100 is a shunting device, the device 100 further includes a central lumen (not shown) extending through the first anchoring portion 110, the second anchoring portion 120, and the intermediate portion 115 to permit fluid to flow therethrough. In such embodiments, the first anchoring portion 110 and the second anchoring portion 120 form generally toroidal-shaped chambers 114, 124 to accommodate the lumen. A representative example of a shunt that can be formed in accordance with the present technology is an interatrial shunt configured to fluidly connect a right atrium and a left atrium to shunt blood therebetween. Examples of interatrial shunts that can incorporate the present technology are described in International Patent Application No. PCT/US2020/063360, the disclosure of which is incorporated by reference herein in its entirety and for all purposes.

Although the device 100 is shown as having a first chamber 114 and a second chamber 124, in some embodiments, the device 100 only includes a single chamber. For example, the first anchoring portion 110 can include the first chamber 114, and the second anchoring portion 120 can be omitted, or can be formed of a relatively flat flange-like structure that does not form an enclosed chamber. Additionally or alternatively, the first anchoring portion 110 and the second anchoring portion 120 can have the same or different sizes and/or properties, depending on the desired application of the device. Further, either or both of first chamber 114 and second chamber 124 can have sub-compartments or sub-chambers, any one of which can remain empty or can hold one or more canisters. Accordingly, one skilled in the art will appreciate that the present technology is not limited to the embodiments expressly described herein, but rather is intended to encompass alterations and variations of the embodiments described herein.

### Examples

Several aspects of the present technology are set forth in the following examples:
1. An implantable medical device, comprising:
a first anchoring portion formed by one or more first filaments, the first anchoring portion defining a first internal chamber;
a second anchoring portion formed by one or more second filaments, the second anchoring portion defining a second internal chamber;
an intermediate portion connecting the first anchoring portion and the second anchoring portion; and
one or more canisters positioned within and retained by the first internal chamber and/or the second internal chamber, wherein the one or more canisters include a fluidly sealed housing that carries one or more components of the medical device.
2. The device of example 1 wherein the one or more components carried in the housing comprises one or more energy storage components.
3. The device of examples 1 or 2 wherein the one or more components carried in the housing comprises one or more sensors.
4. The device of any of examples 1-3 wherein the one or more components carried in the housing comprises electronics.
5. The device of any of examples 1-4 wherein the one or more canisters include at least a first canister positioned in the first internal chamber and at least a second canister positioned in the second internal chamber.
6. The device of any of examples 1-5 wherein the one or more canisters are connected to the one or more first filaments and/or the one or more second filaments.
7. The device of any of examples 1-5 wherein the one or more canisters are not connected to the one or more first filaments and/or the one or more second filaments.
8. The device of any of examples 1-7 wherein the one or more first filaments include one or more first braided wires, and wherein the one or more second filaments include one or more second braided wires.
9. The device of any of examples 1-8 wherein the one or more canisters include at least a first canister and a second canister positioned in the first internal chamber, wherein the first canister and the second canister are coupled.
10. The device of example 9 wherein the first canister and the second canister are coupled via an electrical lead.
11. The device of any of examples 1-10, further comprising a lumen extending through the first anchoring portion, the second anchoring portion, and the intermediate portion, wherein, when the device is implanted in a patient, the lumen is configured to shunt fluid.
12. The device of any of example 1-10 wherein:
the first anchoring portion includes a first membrane coupled to the one or more first filaments and at least partially enclosing the first internal chamber, and
the second anchoring portion includes a second membrane coupled to the one or more second filaments and at least partially enclosing the second internal chamber.
13. The device of any of examples 1-10 wherein:
the first internal chamber is configured to be fluidly coupled to an environment external to the first anchoring portion, and
the second internal chamber is configured to be fluidly coupled to an environment external to the second anchoring portion.
14. An implantable medical device, comprising:
a first anchoring portion defining a first internal chamber;
a second anchoring portion defining a second internal chamber;
an intermediate portion connecting the first anchoring portion and the second anchoring portion; and
one or more canisters positioned within and retained by the first internal chamber and/or the second internal chamber,
wherein the one or more canisters include a fluidly sealed housing that carries one or more components of the medical device, and
wherein the first anchoring portion, the second anchoring portion, and the intermediate portion are formed at least partially by one or more filaments.
15. The device of example 14 wherein the first anchoring portion, the second anchoring portion, and the intermediate portion are formed by a single filament.
16. The device of example 15 wherein the single filament is a braided wire.
17. The device of example 14 wherein the first anchoring portion, the second anchoring portion, and the intermediate portion are formed by at least two filaments coupled together.
18. The device of any of examples 14-17 wherein the one or more filaments are composed of Nitinol.
19. The device of any of examples 14-18 wherein the one or more components include one or more energy storage components, one or more sensors, and/or electronics.
20. The device of any of examples 14-19, further comprising a lumen extending through the first anchoring portion, the second anchoring portion, and the intermediate portion, and wherein, when the device is implanted in a patient, the lumen is configured to shunt fluid therethrough.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An implantable medical device, comprising:
a first anchoring portion formed by one or more first filaments, the first anchoring portion defining a first internal chamber;
a second anchoring portion formed by one or more second filaments, the second anchoring portion defining a second internal chamber;
an intermediate portion connecting the first anchoring portion and the second anchoring portion; and
one or more canisters positioned within and retained by the first internal chamber and/or the second internal chamber, wherein the one or more canisters include a fluidly sealed housing that carries one or more components of the medical device.

2. The device of claim 1 wherein the one or more components carried in the housing comprises one or more energy storage components.

3. The device of claim 1 wherein the one or more components carried in the housing comprises one or more sensors.

4. The device of claim 1 wherein the one or more components carried in the housing comprises electronics.

5. The device of claim 1 wherein the one or more canisters include at least a first canister positioned in the first internal chamber and at least a second canister positioned in the second internal chamber.

6. The device of claim 1 wherein the one or more canisters are connected to the one or more first filaments and/or the one or more second filaments.

7. The device of claim 1 wherein the one or more canisters are not connected to the one or more first filaments and/or the one or more second filaments.

8. The device of claim 1 wherein the one or more first filaments include one or more first braided wires, and wherein the one or more second filaments include one or more second braided wires.

9. The device of claim 1 wherein the one or more canisters include at least a first canister and a second canister positioned in the first internal chamber, wherein the first canister and the second canister are coupled.

10. The device of claim 9 wherein the first canister and the second canister are coupled via an electrical lead.

11. The device of claim 1, further comprising a lumen extending through the first anchoring portion, the second anchoring portion, and the intermediate portion, wherein, when the device is implanted in a patient, the lumen is configured to shunt fluid.

12. The device of claim 1 wherein:
the first anchoring portion includes a first membrane coupled to the one or more first filaments and at least partially enclosing the first internal chamber, and
the second anchoring portion includes a second membrane coupled to the one or more second filaments and at least partially enclosing the second internal chamber.

13. The device of claim 1 wherein:
the first internal chamber is configured to be fluidly coupled to an environment external to the first anchoring portion, and
the second internal chamber is configured to be fluidly coupled to an environment external to the second anchoring portion.

14. An implantable medical device, comprising:
a first anchoring portion defining a first internal chamber;
a second anchoring portion defining a second internal chamber;
an intermediate portion connecting the first anchoring portion and the second anchoring portion; and
one or more canisters positioned within and retained by the first internal chamber and/or the second internal chamber,
wherein the one or more canisters include a fluidly sealed housing that carries one or more components of the medical device, and
wherein the first anchoring portion, the second anchoring portion, and the intermediate portion are formed at least partially by one or more filaments.

15. The device of claim 14 wherein the first anchoring portion, the second anchoring portion, and the intermediate portion are formed by a single filament.

16. The device of claim 15 wherein the single filament is a braided wire.

17. The device of claim 14 wherein the first anchoring portion, the second anchoring portion, and the intermediate portion are formed by at least two filaments coupled together.

18. The device of claim 14 wherein the one or more filaments are composed of Nitinol.

19. The device of claim 14 wherein the one or more components include one or more energy storage components, one or more sensors, and/or electronics.

20. The device of claim 14, further comprising a lumen extending through the first anchoring portion, the second anchoring portion, and the intermediate portion, and wherein, when the device is implanted in a patient, the lumen is configured to shunt fluid therethrough.
